(19) United States
(12) Patent Application Publication
(10) Pub. No.: US 2024/0169684 A1
(43) Pub. Date: May 23, 2024

(54) LASER SPECKLE CONTRAST IMAGING SYSTEM AND LASER SPECKLE CONTRAST IMAGING METHOD THEREOF

(71) Applicant: National Health Research Institutes, Miaoli County (TW)

(72) Inventors: Lun-De Liao, Miaoli County (TW); Sheng-Tong Dai, Miaoli County (TW)

(21) Appl. No.: 18/518,452

(22) Filed: Nov. 23, 2023

(30) Foreign Application Priority Data
Nov. 23, 2022 (TW) 111144830

(51) Int. Cl. G06V 10/25; H04N 23/21; H04N 23/56

(52) U.S. Cl. CPC G06V 10/25; H04N 23/21; H04N 23/56; A61B 5/0261

(57) ABSTRACT

A laser speckle contrast imaging system includes a laser beam, configured to emit an object; an image capturing module, configured to capture an image data of the object; and a processing unit, coupled to the laser beam and the image capturing module, configured to generate a first image and a second image corresponding to the laser beam according to the image data, and to perform an auto-tracking function for an interest of region (ROI) of the second image and update the second image according to an auto-tracking result of the ROI.

### BACKGROUND OF THE INVENTION

### 1. Field of the Invention

The present invention relates to a laser speckle contrast imaging system and a related laser speckle contrast imaging method, and more particularly, to a laser speckle contrast imaging system and a related laser speckle contrast imaging method capable of performing an auto-tracking function.

### 2. Description of the Prior Art

State of the microcirculation of blood is an index for healing ability. Laser Doppler imaging (LDI) is a common method for handling burn injuries clinically, which visualizes the blood flow. However, disadvantages of the LDI are high cost, huge device volume and long collection time. On the other hand, laser speckle contrast imaging (LSCI) systems may implement similar results and have advantages of low cast, shorter collection time. In addition, the LSCI systems are non-intrusive and contactless.

However, the conventional blood laser detecting instrument cannot be easily moved to detect an injured part of the patient. When a patient moves, recollection of images of the injured part is required, which causes inconveniences to the patient. Further, a conversion efficiency of electronic-to-optical is around 50% of the conventional laser diode, which means that 50% of electric energy is converted to thermal energy. Therefore, when the laser diode is turned on, the temperature of the laser diode is increased gradually, the energy cannot be output stably, and the lifetime of the laser diode is shortened with the increased temperature.

Therefore, improvements are necessary to the conventional technique.

### SUMMARY OF THE INVENTION

In light of this, the present invention provides a laser speckle contrast imaging system and a related laser speckle contrast imaging method to perform an auto-tracking function and to improve a user experience.

An embodiment of the present invention discloses a laser speckle contrast imaging system, comprises a laser beam, configured to emit an object; an image capturing module, configured to capture an image data of the object; and a processing unit, coupled to the laser beam and the image capturing module, configured to generate a first image and a second image corresponding to the laser beam according to the image data, and to perform an auto-tracking function for an interest of region (ROI) of the second image and update the second image according to an auto-tracking result of the ROI.

Another embodiment of the present invention discloses a laser speckle contrast imaging method, for a laser speckle contrast imaging system, wherein the laser speckle contrast imaging system includes a laser beam, an image capturing module and a processing unit, and the laser speckle contrast imaging method comprises emitting, by the laser beam, an object capturing, by the image capturing module, an image data of the object; generating, by the processing unit, a first image and a second image corresponding to the laser beam according to the image data; and performing, by the processing unit, an auto-tracking function for an interest of region (ROI) of the second image and updating the second image according to an auto-tracking result of the ROI.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is a schematic diagram of a laser speckle contrast imaging (LSCI) system according to an embodiment of the present invention.

FIG. 2 is a schematic diagram of a user interface of the LSCI system according to an embodiment of the present invention.

FIG. 3 is a schematic diagram of an auto-tracking function for an interest of region (ROI) according to an embodiment of the present invention.

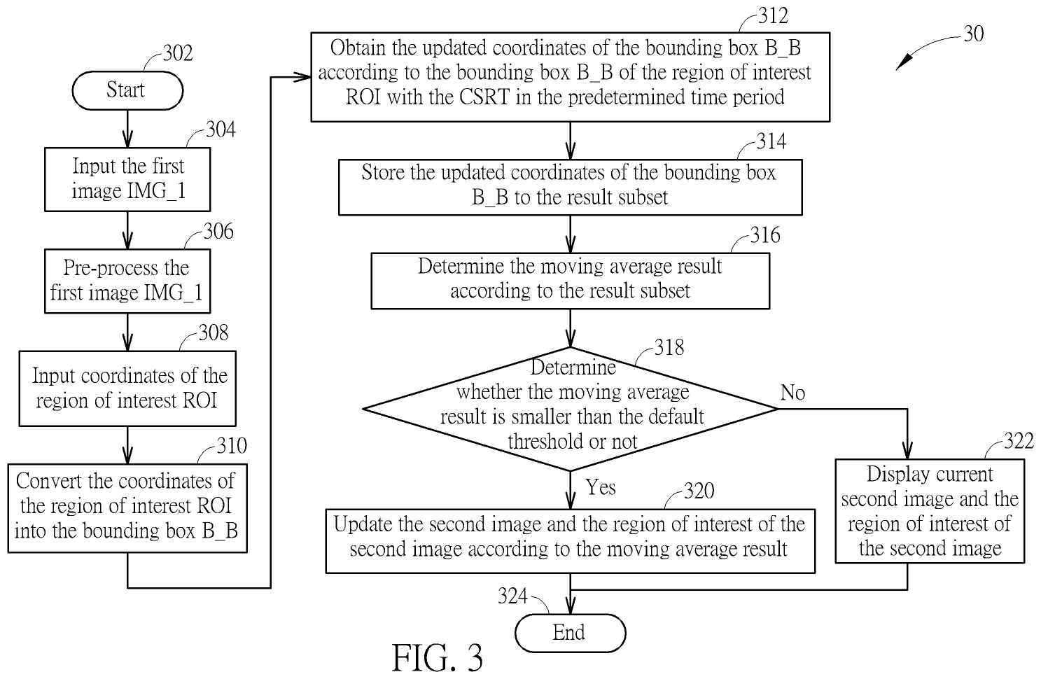

FIG. 4 is a schematic diagram of a heat dissipating device according to an embodiment of the present invention.

### DETAILED DESCRIPTION

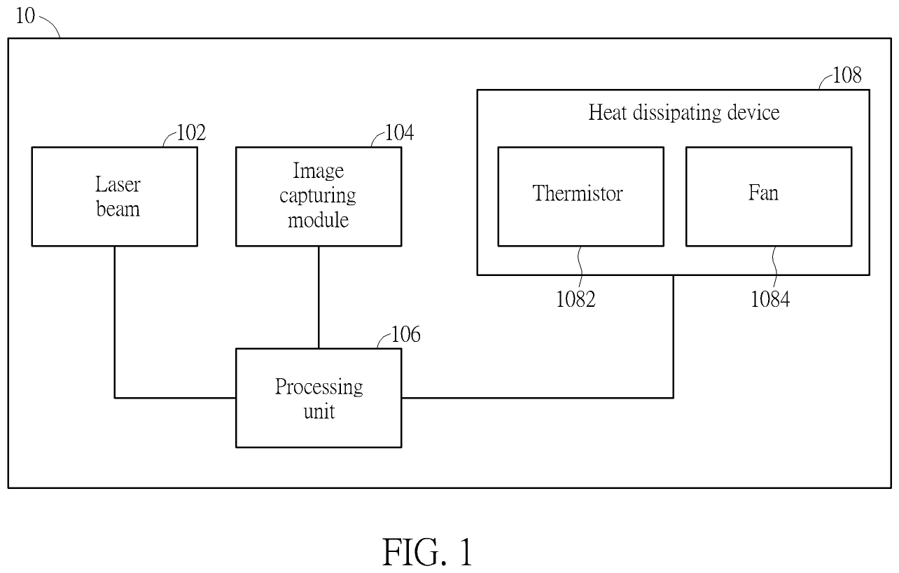

Please refer to FIG. 1, which is a schematic diagram of a laser speckle contrast imaging (LSCI) system 10 according to an embodiment of the present invention. The laser speckle contrast imaging system 10 may be utilized for monitoring microcirculation of a subject, which includes a laser beam 102, an image capturing module 104, a processing unit 106 and a heat dissipating device 108. The laser beam 102 is utilized for emitting an object, e.g. an injured part of the subject. The image capturing module 104 may be a near-infrared image capturing module, for capturing an image data of the object. The processing unit 106 may be a central processor or a device with processing functions. The processing unit 106 is coupled to the image capturing module 104, and is configured to generate a first image and a second image corresponding to the laser beam according to the image data, wherein the processing unit 106 is configured to perform an auto-tracking function for an interest of region (ROI) of the second image and to update the second image according to an auto-tracking result of the ROI. The heat dissipating device 108 includes a thermistor 1082 and a fan 1084, wherein the thermistor 1082 is utilized for measuring a temperature of a laser diode of the laser beam 102 to adjust a rotating speed of the fan 1084 and to adjust the temperature of the laser beam 102.

In an embodiment, the first image may be an image captured by the near-infrared image capturing module, the second image may be a laser speckle contrast image emitted by the laser beam 102. In addition, the auto-tracking function may be executed by a channel spatial reliability-discriminative correlation filter (DCF-CSR), such that the laser speckle contrast imaging system 10 according to an embodiment of the present invention may automatically track the injured part of a patient and the user experience (UX) is improved.

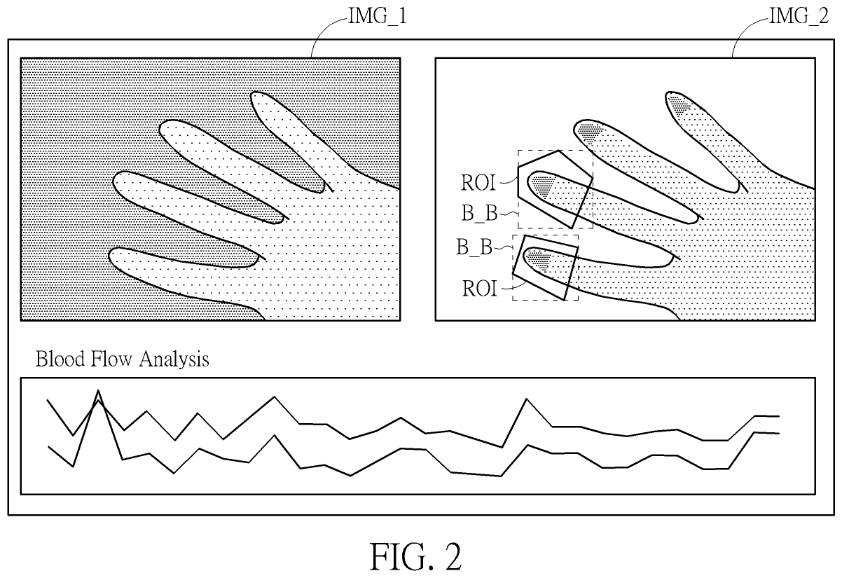

Please refer to FIG. 2, which is a schematic diagram of a user interface (UI) of the LSCI system 10 according to an embodiment of the present invention. FIG. 2 shows the image data processed by the processing unit 106, i.e. the first image IMG_1 and the second image IMG_2, and an operation interface for the user, e.g. blood flow analysis and analysis of region of interest ROI.

In an example, when the laser speckle contrast imaging system 10 according to an embodiment of the present invention is in operation, an injured part, e.g. a palm, of the patient is placed within a capturing range of the image capturing module 104 of the laser speckle contrast imaging system 10, the first image IMG_1 and the second image IMG_2 are displayed as shown in FIG. 2.

The user may select at least a region of interest ROI in the second image IMG_2 through the user interface of FIG. 2, such that the processing unit 106 of the laser speckle contrast imaging system 10 according to an embodiment of the present invention may automatically track the injured part of the patient according to the selected region of interest ROI. Notably, the region of interest ROI may be a random or default polygon or circle, but not limited thereto.

The processing unit 106 is configured to determine a bounding box B_B according to the region of interest ROI, and the bounding box B_B is formed by a maximal horizontal axis value x_max, a minimal horizontal axis value x_min, a maximal vertical axis value y_max and a minimal vertical axis value y_min of the ROI of the second image IMG_2. For example, when the region of interest ROI is a pentagon, the processing unit 106 may determine coordinates of the bounding box B_B as (x_max, y_max), (x_min, y_max), (x_max, y_min), (x_min, y_min) according to the pentagon.

Since the laser speckle contrast imaging system 10 according to an embodiment of the present invention processes the first image IMG_1 and the second image IMG_2 in real-time, the processing unit 106 may perform the auto-tracking function for the region of interest ROI with a dynamic programming method to determine a channel spatial reliability tracker (CSRT) of the DCF-CSR, such that the processing unit 106 may enlarge and position the region of interest ROI with a relatively low frame per second (FPS), e.g. 11-25 FPS, to achieve a better efficiency.

After the processing unit 106 determines the bounding box B_B according to the region of interest ROI, and then determines an updated bounding box B_B according to a previous second image, the current second image IMG_2, an ROI of the previous second image, and the ROI of the second image IMG_2 with the CSRT. In an embodiment, since the updated bounding box B_B is a rectangle, and assume that the minimal horizontal axis value x_min and the maximal vertical axis value y_max of each image are zero, i.e. an origin of a coordinate, the processing unit 106 may record the updated maximal horizontal axis value x_max and the updated maximal vertical axis value y_max of the rectangular bounding box B_B to obtain all updated coordinates.

The processing unit 106 is configured to collect a result subset having a plurality of updated bounding boxes, e.g. ten sets of updated bounding boxes B_B, in a predetermined time period, e.g. within one second, and to determine a moving average result according to a moving average of the result subset.

Therefore, the processing unit 106 may compare the moving average result to a default threshold to determine whether any outlier exists in the result subset of the updated bounding box B_B. Specifically, the above steps may be utilized for filtering abnormal coordinates of the result subset, e.g. when the moving average of the result subset is larger than or equal to 1.5 times (i.e. the default threshold) of a maximal value of the horizontal axis or the vertical axis of the second image IMG_2, the second image IMG_2 and the region of interest ROI of the second image IMG_2 are not updated by the processing unit 106, i.e. the current second image IMG_2 is displayed; in contrast, when the moving average of the result subset is smaller than 1.5 times of a maximal value of the horizontal axis or the vertical axis of the second image IMG_2, the second image IMG_2 and the region of interest ROI of the second image IMG_2 are updated according to the moving average result by the processing unit 106.

Assume that the processing unit 106 sets coordinates of the second image IMG_2 on a coordinate system as (0, 0), (100, 0), (0, 100), (100, 100). When the coordinates of the moving average result determined by the CSRT of the processing unit 106 in 1 second is (160, 160), which represents that the moving average result of the second image IMG_2 is larger than the default threshold, the second image IMG_2 and the region of interest ROI of the second image IMG_2 are not updated by the processing unit 106, i.e. display the current second image IMG_2; in contrast, when the coordinates of the moving average result determined by the CSRT of the processing unit 106 in 1 second is (90, 90), which represents that the moving average result of the second image IMG_2 is smaller than the default threshold, the second image IMG_2 and the region of interest ROI of the second image IMG_2 are updated according to the moving average result by the processing unit 106.

Therefore, the laser speckle contrast imaging system 10 according to an embodiment of the present invention may auto-track the injured part when measuring, and the region of interest ROI is correspondingly moved without manual adjustment, which substantially decreases the measurement time and improves the measurement efficiency.

The above operations of auto-tracking the region of interest ROI of the laser speckle contrast imaging system 10 may be summarized as an auto-tracking method 30 for the region of interest ROI. The auto-tracking method 30 for the ROI includes the following steps:
Step 302: Start;
Step 304: Input the first image;
Step 306: Pre-process the first image;
Step 308: Input coordinates of the region of interest ROI;
Step 310: Convert the coordinates of the region of interest ROI into the bounding box B_B;
Step 312: Obtain the updated coordinates of the bounding box B_B according to the bounding box B_B of the region of interest ROI with the CSRT in the predetermined time period;
Step 314: Store the updated coordinates of the bounding box B_B to the result subset;
Step 316: Determine the moving average result according to the result subset;
Step 318: Determine whether the moving average result is smaller than the default threshold or not, if yes, goes to step 320; if no, goes to step 322;
Step 320: Update the second image and the region of interest of the second image according to the moving average result;
Step 322: Display current second image and the region of interest of the second image; Step 324: End.

Regarding operations of the auto-tracking method 30 for the region of interest ROI, please refer to the above mentioned embodiments of the LSCI system 10, which is not narrated herein again for brevity.

Notably, the pre-processing of the first image in step 306 may include normalizing the first image. In an embodiment, since the CSRT tracking technique aims for processing the images of 8 bits, and the first image may be an NIR image of 16 bits, the first image is converted in step 306 to prevent the degradation of the image quality.

On the other hand, since the first image IMG_1 and the second image IMG_2 are correspondingly displayed in FIG. 2, the identical coordinate system and coordinates are shared. The user may select the region of interest ROI on the first image IMG_1 or the second image IMG_2 according to different settings of the laser speckle contrast imaging system 10, and is not limited to the embodiment illustrated in FIG. 2.

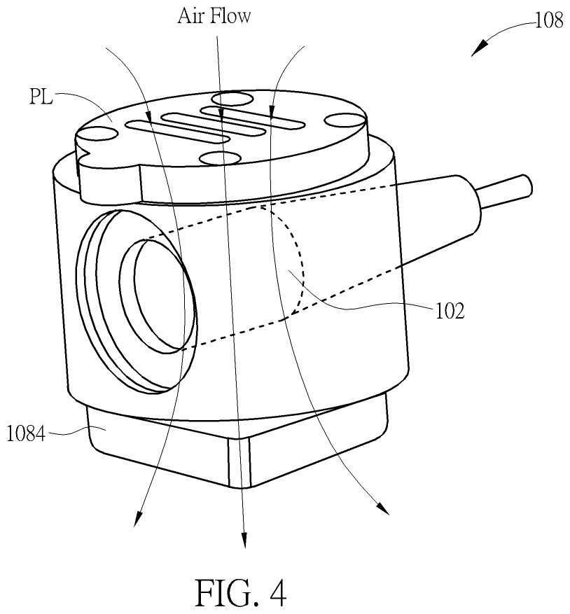

In addition, the heat dissipating device 108 of the laser speckle contrast imaging system 10 according to an embodiment of the present invention is utilized for alleviating a heat dissipation effect of the laser beam 102. Please refer to FIG. 4, which is a schematic diagram of the heat dissipating device 108 according to an embodiment of the present invention. The thermistor 1082, which is not illustrated in the figure, of the heat dissipating device 108 of the laser speckle contrast imaging system 10 is a measuring device, which measures a temperature of the laser diode of the laser beam 102, such that the processing unit 106 may compare the temperature target of the laser diode and a temperature with a proportional-integral-derivative (PID) controller, and a rotation speed of the fan 1084 is controlled to speed up to improve the heat dissipation efficiency when the temperature of the laser diode is higher than the target temperature.

In FIG. 4, air circulation is formed by a protective lid PL and the fan 1084 of the heat dissipating device 108 to achieve the heat dissipation and to stabilize the laser energy and the strength of a contrast imaging value, such that a probability of misdiagnosis is decreased and a practicality is improved.

On the other hand, in order to attain perfection the application in clinical practice, as shown in FIG. 2, the laser speckle contrast imaging system 10 may be integrated with other operational functions, e.g. a switch of the NIR camera, adjustment of the default threshold of the LSCI and control switches of the laser beam to improve related operations for the user experience.

In addition, the FPS of the image capturing module 104 is set as above 25 for the convenience of calibration and the energy strength. And the processing unit 106 according to an embodiment of the present invention is executed with a parallel computing method to improve the FPS of the image capturing module 104 and the efficiency of the user interface.

Notably, those skilled in the art may make proper modifications to a laser speckle contrast imaging system and a related laser speckle contrast imaging method of the present invention according to different system requirements. For example, the default threshold for the moving average result, the determination of the region of interest and the predetermined time period of collecting the bounding box may be adjusted according to different requirements, which are not limited thereto. These modifications all belong to the scope of the present invention.

In summary, the present invention provides a laser speckle contrast imaging system and a related laser speckle contrast imaging method, which performs an auto-tracking function for images with an injured part of a patient to decrease a measuring time, stabilizes a temperature of a laser diode with a heat dissipating device and integrates UI/UX interface with a parallel computing method to improve a user experience.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A laser speckle contrast imaging system, comprising:
a laser beam, configured to emit an object;
an image capturing module, configured to capture an image data of the object; and
a processing unit, coupled to the laser beam and the image capturing module, configured to generate a first image and a second image corresponding to the laser beam according to the image data, and to perform an auto-tracking function for an interest of region (ROI) of the second image and update the second image according to an auto-tracking result of the ROI.

2. The laser speckle contrast imaging system of claim 1, wherein the auto-tracking function is performed based on a reliability and a discriminative correlation filter with channel and spatial reliability (DCF-CSR).

3. The laser speckle contrast imaging system of claim 2, wherein the ROI is configured to determine a bounding box, and the bounding box is formed by a maximal horizontal axis value, a minimal horizontal axis value, a maximal vertical axis value and a minimal vertical axis value of the ROI.

4. The laser speckle contrast imaging system of claim 2, wherein the processing unit is configured to perform the auto-tracking function for the ROI with a dynamic programming method to determine a channel spatial reliability tracker (CSRT) of the DCF-CSR.

5. The laser speckle contrast imaging system of claim 4, wherein the processing unit is configured to collect a result subset having a plurality of updated bounding boxes in a predetermined time period, and to determine a moving average result according to a moving average of the result subset.

6. The laser speckle contrast imaging system of claim 5, wherein each of the plurality of updated bounding boxes is determined by the processing unit according to a previous second image, the second image, an ROI of the previous second image, and the ROI of the second image.

7. The laser speckle contrast imaging system of claim 5, wherein the processing unit is configured to update the second image and the ROI corresponding to the second image according to the moving average result of the second image.

8. The laser speckle contrast imaging system of claim 1, wherein the processing unit is executed with a parallel computing method.

9. The laser speckle contrast imaging system of claim 1, further comprising:
a heat dissipating device, including a thermistor and a fan, wherein the thermistor is configured to measure a temperature of a laser diode of the laser beam to adjust a rotating speed of the fan;
wherein the processing unit is configured to compare a target temperature and the temperature of the laser diode with a proportional-integral-derivative (PID) controller to adjust the rotating speed of the fan.

10. The laser speckle contrast imaging system of claim 1, wherein the image capturing module is a near-infrared image capturing module.

11. A laser speckle contrast imaging method, for a laser speckle contrast imaging system, wherein the laser speckle contrast imaging system includes a laser beam, an image capturing module and a processing unit, and the laser speckle contrast imaging method comprising:
emitting, by the laser beam, an object
capturing, by the image capturing module, an image data of the object;
generating, by the processing unit, a first image and a second image corresponding to the laser beam according to the image data; and
performing, by the processing unit, an auto-tracking function for an interest of region (ROI) of the second image and updating the second image according to an auto-tracking result of the ROI.

12. The laser speckle contrast imaging method of claim 11, wherein the auto-tracking function is performed based on a reliability and a discriminative correlation filter with channel and spatial reliability (DCF-CSR).

13. The laser speckle contrast imaging method of claim 12, wherein the ROI is configured to determine a bounding box, and the bounding box is formed by a maximal horizontal axis value, a minimal horizontal axis value, a maximal vertical axis value and a minimal vertical axis value of the ROI.

14. The laser speckle contrast imaging method of claim 12, wherein the processing unit is configured to perform the auto-tracking function for the ROI with a dynamic programming method to determine a channel spatial reliability tracker (CSRT) of the DCF-CSR.

15. The laser speckle contrast imaging method of claim 14, wherein the processing unit is configured to collect a result subset having a plurality of updated bounding boxes in a predetermined time period, and to determine a moving average result according to a moving average of the result subset.

16. The laser speckle contrast imaging method of claim 15, wherein each of the plurality of updated bounding boxes is determined by the processing unit according to a previous second image, the second image, an ROI of the previous second image, and the ROI of the second image.

17. The laser speckle contrast imaging method of claim 15, wherein the processing unit is configured to update the second image and the ROI corresponding to the second image according to the moving average result of the second image.

18. The laser speckle contrast imaging method of claim 11, wherein the processing unit is executed with a parallel computing method.

19. The laser speckle contrast imaging method of claim 11, wherein laser speckle contrast imaging system further comprises a heat dissipating device, including a thermistor and a fan, wherein the thermistor is configured to measure a temperature of a laser diode of the laser beam to adjust a rotating speed of the fan; and the processing unit is configured to compare a target temperature and the temperature of laser the diode with a proportional-integral-derivative (PID) controller to adjust the rotating speed of the fan.

20. The laser speckle contrast imaging method of claim 11, wherein the image capturing module is a near-infrared image capturing module.
